(19) United States
(12) Patent Application Publication
(10) Pub. No.: US 2009/0030278 A1
(43) Pub. Date: Jan. 29, 2009

(54) ELECTRICAL POWER SUPPLY DEVICE FOR ENDOSCOPE AND ENDOSCOPE

(75) Inventor: Tadashi Minakuchi, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/175,795

(22) Filed: Jul. 18, 2008

(30) Foreign Application Priority Data
Jul. 23, 2007 (JP) 2007-191200

(51) Int. Cl. A61B 1/12; A61B 1/06

(52) U.S. Cl. 600/118; 600/178

(57) ABSTRACT

An electrical power supply device is for an endoscope having a scope for insertion into the body of a subject. The electrical power supply device includes a temperature controller and a thermoelectric converter. The thermoelectric converter controls the temperature of the interior of the scope. The thermoelectric converter generates electricity based on the difference in temperature between the body interior of the subject and the interior of the scope.

### BACKGROUND OF THE INVENTION

1. Field of the Invention

The present invention relates to an electrical power supply device for an endoscope, and an endoscope.

2. Description of the Related Art

Usually, an electronic endoscope has a scope including an imaging device and a processor that processes image signals generated by the imaging device. Known endoscopes include one in which electrical power is supplied via a power line from a power circuit provided in a processor to an imaging device in a scope, or another one in which electromagnetic coupling is used for electrical power supply to an imaging device instead of using the power line.

Furthermore, there is also known an electronic endoscope containing in the scope, a solar cell which supplies electrical energy to an imaging device using part of the light for illuminating a subject.

When a power line is connected to a scope, the power line may act as an antenna that transmits or receives external noise. As a result, the quality or a subject image may be degraded, or the imaging device may malfunction. Furthermore, when power is supplied by a power line or by electromagnetic coupling, the diameter of the insertion portion of a scope which is inserted into the body of a subject may increase.

On the other hand, in an electronic endoscope where a part of the illuminating light for illuminating a subject is used unaltered as a power source, some useful illuminating light is lost and it is impossible to effectively use all the illuminating light emitted from a light source for observing a subject.

### SUMMARY OF THE INVENTION

Therefore, an objective of the present invention is to provide an electrical power supply device for an endoscope, that eliminates noise, reduces the diameter of the insertion portion of a scope, and allows the effective utilization of the illuminating light.

A first electrical power supply device according to the present invention, is for an endoscope having a scope for insertion into the body of a subject. The first electrical power supply device includes a temperature controller and a thermoelectric converter. The thermoelectric converter controls the temperature of the interior of the scope. The thermoelectric converter generates electricity based on the difference in temperature between the body interior of the subject and the interior of the scope.

The temperature controller may control the temperature of the interior of the scope using the infrared component of illuminating light used to illuminate the body interior of the subject.

The first electrical power supply device may also include a light splitter that splits the illuminating light. The temperature controller may include a photothermal converter that generates heat based on the infrared component that is split by the light splitter. The first electrical power supply device may also include a light-electric converter that generates electricity using part of the illuminating light.

The endoscope may also include an optical fiber to transmit the infrared component to the scope. The first electrical power supply device may also include a light splitter that splits the illuminating light, and the light splitter may be arranged close to the output end of the optical fiber.

The first electrical power supply device may also include a secondary battery that is charged by the electricity generated by the thermoelectric converter.

The secondary battery may be arranged inside the scope and may be chargeable by electromagnetic coupling with the exterior of the scope.

The temperature controller may include a cooler that cools the interior of the scope. The temperature controller may include a photothermal converter that generates heat from light and a light supply that supplies light to the photothermal converter.

A second electrical power supply device according to the present invention is for an endoscope having a scope for insertion into the body of a subject. The second electrical power supply device includes first and second temperature controllers, and a thermoelectric converter. The first temperature controller generates heat based on the infrared component of illuminating light used to illuminate the body interior of the subject. The second temperature controller cools the interior of the scope. The thermoelectric converter generates electricity based on the difference in temperature between the body interior of the subject and the interior of the scope.

A first endoscope according to the present invention includes a scope for insertion into the body of a subject, an imaging device, a temperature controller, and a thermoelectric converter. The temperature controller controls the temperature of the interior of the scope. The thermoelectric converter generates electricity based on the difference in temperature between the body interior of the subject and the interior of the scope. The imaging device is driven by the electricity generated by the thermoelectric converter.

A second endoscope according to the present invention includes a scope for insertion into the body of the subject, an imaging device, first and second temperature controller, and a thermoelectric converter. The first temperature controller generates heat based on the infrared component of illuminating light for illuminating the body interior of the subject. The second temperature controller cools the interior of the scope. The thermoelectric converter generates electricity based on the difference in temperature between the body interior of the subject and the interior of the scope. The imaging device is driven by the electricity generated by the thermoelectric converter.

### BRIEF DESCRIPTION OF THE DRAWINGS

The present invention will be better understood from the description of the preferred embodiments of the invention set forth below together with the accompanying drawings, in which:

FIG. 1 represents an endoscope including an electrical power supply device of a first embodiment;

FIG. 2 represents an endoscope including an electrical power supply device of a second embodiment;

FIG. 3 represents an endoscope including an electrical power supply device of a third embodiment;

FIG. 4 represents an endoscope including an electrical power supply device of a fourth embodiment;

FIG. 5 represents an endoscope including an electrical power supply device of a fifth embodiment;

FIG. 6 represents an endoscope including an electrical power supply device of a sixth embodiment;

FIG. 7 represents an endoscope including an electrical power supply device of a seventh embodiment; and

FIG. 8 represents an endoscope of a comparative example.

### DESCRIPTION OF THE PREFERRED EMBODIMENTS

Hereinafter, the preferred embodiments of the present invention are described with reference to the attached drawings.

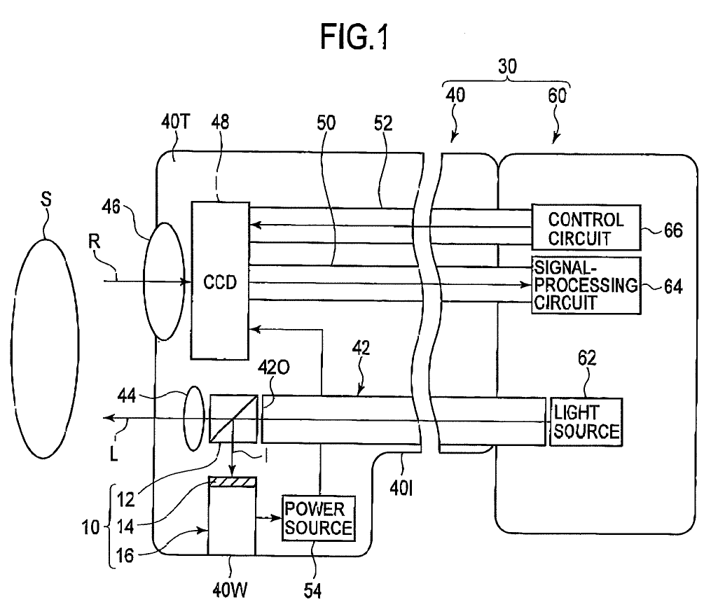

As shown in FIG. 1, an electronic endoscope 30 includes a scope 40 and a processor 60. The scope 40 is inserted into the body of a subject to view a body part S, and is then used. In the processor 60, a light source 62 that emits illuminating light L, is provided. The light source 62, for example, may be a halogen lamp or a xenon lamp, and the wavelength range of the illuminating light L includes the infrared range. That is, the illuminating light L includes an infrared component.

The illuminating light L is transmitted to the tip 40T of the scope 40, via a light-guide fiber (an optical fiber) 42, a lighting lens 44, and other components provided in the scope 40. The subject S is illuminated by the illuminating light L emitted from the tip 40T. Note that the light-guide fiber 42 is, for example, a quartz fiber or a hollow fiber, and the infrared component is also transmitted.

Reflected light R reflected on the subject S enters the tip 40T. In the tip 40T, an object lens 46, and a CCD (imaging device) 48 are provided. In the CCD 48, image signals are generated based on the incidence of reflected light R. Generated image signals are transmitted to the processor 60, via an image-signal transmission channel 50. Image signals are processed in a signal-processing circuit 64 that is provided in the processor 60. As a result, an image of the subject S is generated.

The CCD 48 is controlled by a control circuit 66 provided in the processor 60. That is, the CCD 48 is controlled by signals that are transmitted from the control circuit 66, via a control-signal transmission channel 52.

An electrical power supply device 10 is provided in the tip 40T of the scope 40. The electrical power supply device 10 generates electricity as explained below, so that the electricity to drive the CCD 48 is supplied to the CCD 48 via a power source 54.

The electrical power supply device 10 includes a beam splitter 12 (a light splitter) for splitting the illuminating light L. The beam splitter 12 allows light components including visible light to pass straight but reflects infrared ray I. The infrared ray I that is split by the beam splitter 12 in this way enters an infrared ray absorber 14 (a temperature controller, a photothermal converter, a first temperature controller) included in the electrical power supply device 10.

The infrared ray absorber 14 includes a material which easily absorbs the infrared ray, such as, a carbon black (not shown). Therefore, the infrared ray absorber 14 generates heat by photothermal conversion, when the infrared ray I enters. When the temperature of the interior of the scope 40 increases due to the heat generation by the infrared ray absorber 14, a difference in temperature is created between in the exterior of the tip 40T (that is, the body interior of the subject including a body part S), and the periphery of the electrical power supply device 10 in the interior of the tip 40T. For example, the temperature of the outside wall 40W of the tip 40T is almost equal to that of the body interior of the subject person, such as about 37° C., while the temperature around the infrared ray absorber 14 may reach about 70° due to heat generation.

A peltier device 16 (a thermoelectric converter) is provided in the electrical power supply device 10. The peltier device 16 generates electricity based on the above-explained difference in temperature. The peltier device 16 is arranged to be in contact with the infrared ray absorber 14 and the outside wall 40W of the tip 40T, so that the above-explained temperature difference can be effectively utilized in the generation of electricity by the peltier device 16.

Note that the beam splitter 12 selectively splits the infrared ray I having a wavelength of more than around 700 nm. Therefore, all visible light components included in the illuminating light L emitted by the light source 62 will be effectively used for illuminating the subject S, and the infrared ray I having suitable energy for heat generation is supplied to the infrared ray absorber 14. Furthermore, because the beam splitter 12 is arranged close to the output end 42O of the light-guide fiber 42, effective light splitting is possible.

In the first embodiment, as explained above, electricity is supplied to components such as the CCD 48 in the tip 40T without the use of a power line, so that noise generation caused by the power line can be prevented. Furthermore, although the electrical power supply device 10 should be provided in the tip 40T, the diameter of the insertion portion 40I of the scope 40 for insertion into the body can be reduced, because a power line is not necessary. In addition, all the visible light components can be used for illuminating and photographing the subject S, so that the illuminating light emitted by the light source 62 can be effectively utilized.

Note that electricity generated by the electrical power supply device 10 may not only be used to drive the CCD 48, but also other components such as another circuit in a part provided in the tip 40T. This electrical power supply is applied to the following embodiments as well as the first embodiment.

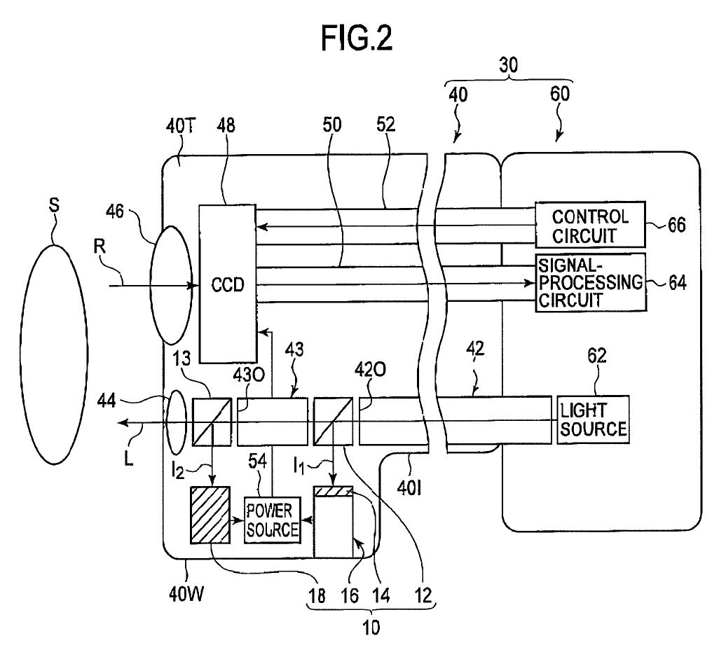

Next, the second embodiment is explained with reference to FIG. 2. Note that in FIG. 2 and those following, components identical to those of the first embodiment are identified by the same numerals.

In the second embodiment, the first and second beam splitters 12 and 13, and a solar cell 18 (a light-electric converter) that generates electricity using the second infrared ray I₂ split from the illuminating light L by the second beam splitter 13, are provided in the electrical power supply device 10, as opposed to the first embodiment. Note that the first beam splitter 12 is arranged between the light-guide fiber 42 and all extended guide fiber 43, and the second beam splitter 13 is arranged close to the output end 43O of the extended guide fiber 43.

As explained above, the second infrared ray I₂ is further split by the second beam splitter 13 from the illuminating light L that has passed the first beam splitter 12 for splitting the first infrared ray I₁, and the second infrared ray I₂ is converted to electrical energy by the solar cell 18. Thus, the electricity generated by the solar cell 18 is also supplied to the power source 54. Accordingly, in the present embodiment, a larger amount of electricity can be supplied to the CCD 48 and other component than in the first embodiment.

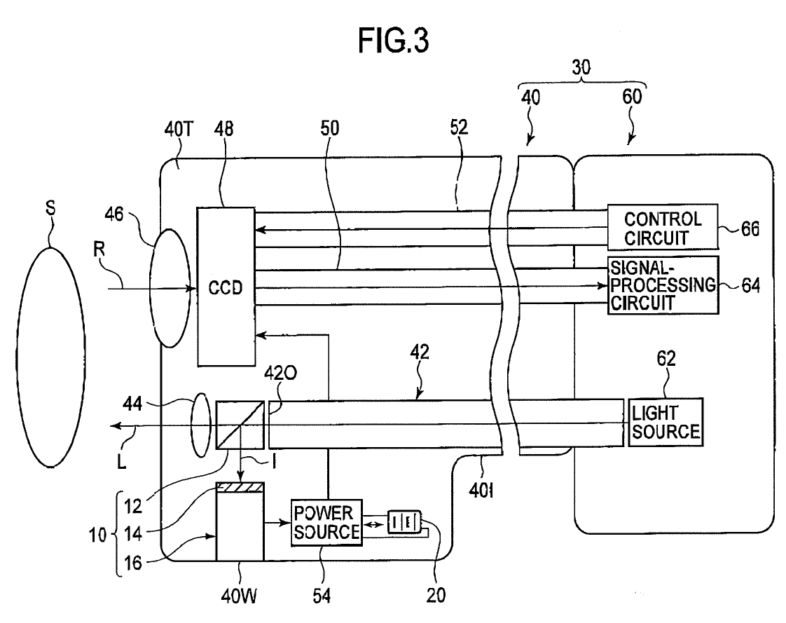

Next, the third embodiment is explained. In the third embodiment, as represented in FIG. 3, a secondary battery 20 that can be charged by the electricity generated by the peltier device 16 is provided in the electrical power supply device 10, differing from the first embodiment. That is, the secondary battery 20 stores excess electricity supplied by the power source 54, and supplies the electricity to the CCD 48 and other components via the power source 54, when required. Therefore, in the present embodiment, more stable electricity is reliably supplied to the CCD 48 and other components, than in the first embodiment.

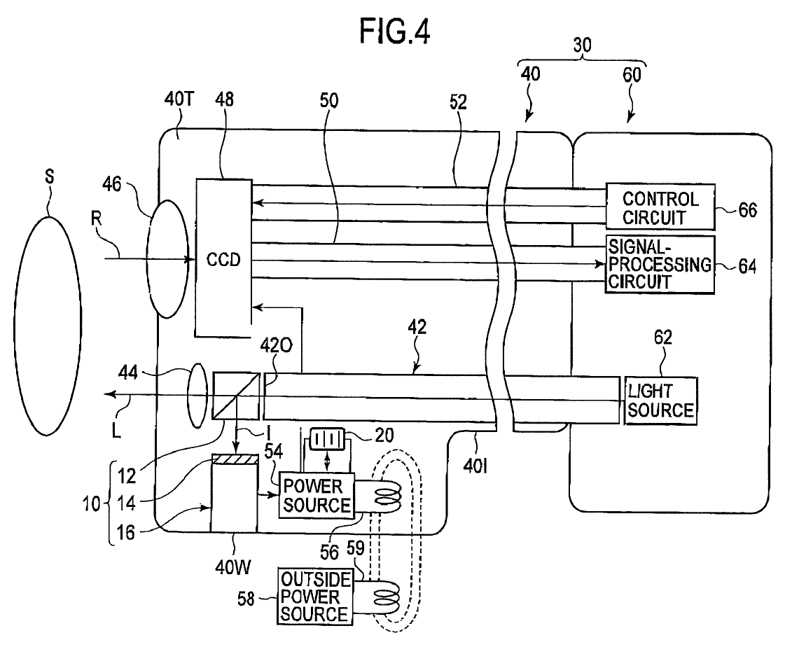

Next, the fourth embodiment is explained with reference to FIG. 4. In the fourth embodiment, the secondary battery 20 can be charged not only by the electricity from the peltier device 16, but also by the exterior of the outside wall 40W of the tip 40T, differing from the third embodiment. That is, a coil 56 is provided in the power source 54, and the secondary battery 20 that is electrically connected to the power source 54 can be charged by the electromagnetic coupling between the coil 56 and an outside coil 59 of an external power source 58.

As explained above, in the fourth embodiment, the secondary battery 20 can be charged using the external power source 58 before the electronic endoscope 30 is used, that is, during the period when the insertion portion 40I of the scope 40 is not inserted into the body of the subject. Accordingly, the CCD 48 and other components can reliably begin functioning even when the emission of the illuminating light L by the light source 62 and electricity generation by the peltier device 16 have only just started, at the starting time of the electronic endoscope 30.

Furthermore, during an operation by a user, the electricity generated by the peltier device 16 can be used as well as the above-explained embodiments. Therefore, although the secondary battery 20 can not be charged by the external power source 58 during operation, observing and photographing the subject S can be carried out without any problem.

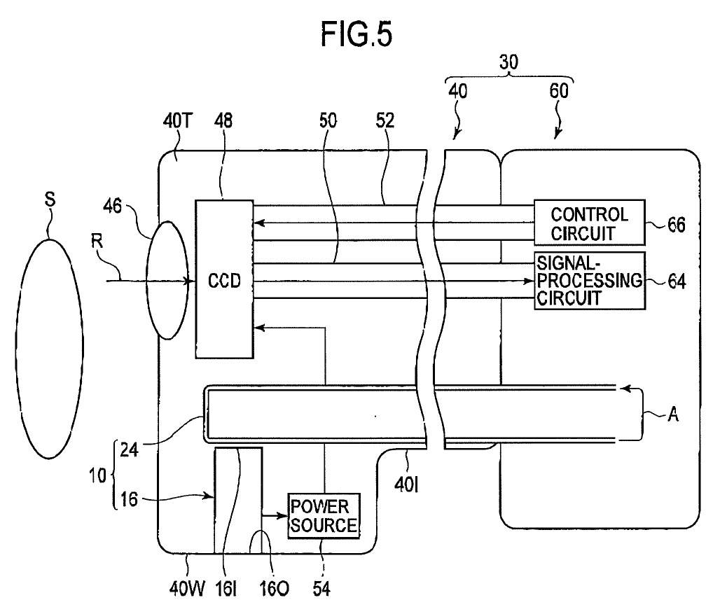

Next, the fifth embodiment is explained with reference to FIG. 5. In the fifth embodiment, the interior temperature of the scope 40 is controlled so that the temperature in the periphery of the peltier device 16 (the interior temperature) is lower than that in the periphery of the outside wall 40W of the tip 40T (the exterior temperature), differing from the above-explained embodiments where the temperature in the periphery of the peltier device 16 is higher than that in the periphery of the outside wall 40W of the tip 40T that is equal to the body temperature of the subject person.

In the present embodiment, because splitting the infrared ray I from the illuminating light L is not necessary, the beam splitter 12 and infrared ray absorber 14 are not provided. Instead of these elements, a coolant circulation channel 24 (a cooler, a second temperature controller) to cool the interior of the scope 40 is provided. In the coolant circulation channel 24, a tube for cooling fluid is provided, so that the cooling fluid is circulated inside the coolant circulation channel 24 by a motor (not shown) provided in the processor 60, as shown by an arrow A.

The outside end 16O of the peltier device 16 contacts the outside wall 40W of the tip 40T, and the inside end 16I thereof is close to the coolant circulation channel 24. Therefore, the deference in temperature between the outside end 16O (close to the body temperature of the subject), and the inside end 16I that is cooled by the cooling fluid in the coolant circulation channel 24, could be as much as 20° C. As a result, the peltier device 16 generates electricity.

Note that the temperature of the cooling fluid circulating in the coolant circulation channel 24 is controlled so as to remain constant by, for example, the processor 30. Water, for example, may be used as the cooling fluid. The water may also be ejected from the tip 40T for use in treating the subject S or for other purposes. Furthermore, by arranging the coolant circulation channel 24 close to, for example, the CCD 48, the temperatures of the CCD 48 or other elements provided in the tip 40T may be controlled. The inside end 16I of the peltier device 16 may contact the coolant circulation channel 24 for efficient electricity generation.

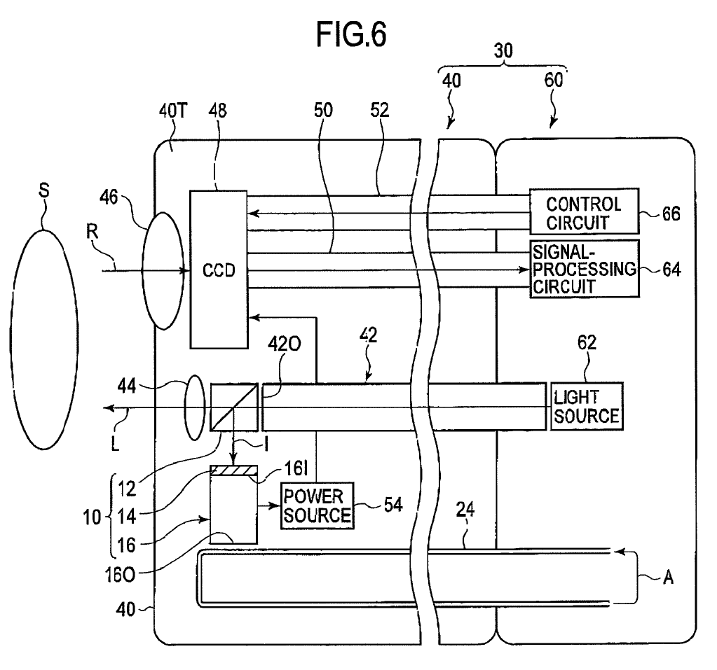

Next, the sixth embodiment is explained with reference to FIG. 6. In the sixth embodiment, the first and fifth embodiments are combined. That is, in the sixth embodiment, as in the first embodiment, the infrared ray included in the illuminating light L is absorbed by the infrared ray absorber 14 (the first temperature controller) and that generates heat, so the temperature of the inside end 16I of the peltier device 16 is increased. Furthermore, as in the fifth embodiment, the outside end 16O of the peltier device 16 is cooled by providing the coolant circulation channel 24 (the second temperature controller). As a result, due to the heat generation at the infrared ray absorber 14, the temperature of the inside end 16I of the peltier device 16 exceeds that of the exterior of the tip 40T (which is close to the body temperature of the subject), and due to the cooling by the coolant circulation channel 24, the temperature of the outside end 16O of the peltier device 16 drops below the body temperature of the subject person. Accordingly, in the present embodiment, the temperature difference will be greater than in the above-explained embodiments.

As explained above, in the present embodiment, the efficiency of generating electricity by the peltier device 16 is increased and the amount of generated electricity increases. Furthermore, just as in the above-explained embodiments, a power line is not provided, so noise can be avoided.

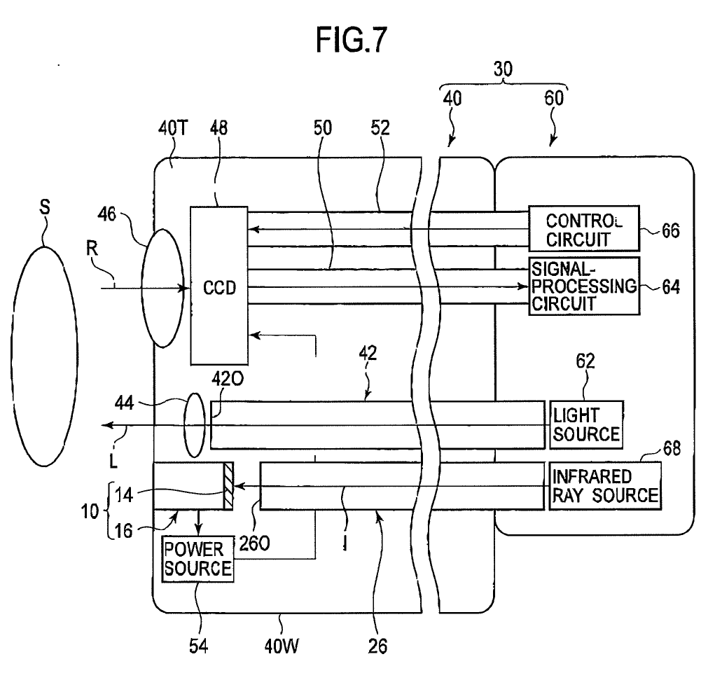

Next, the seventh embodiment is explained with reference to FIG. 7. In the seventh embodiment, the following points are different from the first embodiment. Namely, in the seventh embodiment, the beam splitter 12 is not provided. Provided instead, is an infrared ray guide fiber 26 (a light supply, or a temperature controller) for transmitting infrared ray I to be supplied to the infrared ray absorber 14, from the processor 60 to the scope 40. Furthermore, the arrangement of the infrared ray absorber 14 and the peltier device 16 is changed.

In the present embodiment, the infrared ray guide fiber 26 is independent from the light-guide fiber 42, and the only purpose of the infrared ray guide fiber 26 is the transmission of the infrared ray I emitted by an infrared ray source 68. The infrared ray I exits the output end 26O of the infrared ray guide fiber 26 and enters the infrared ray absorber 14 that is arranged to face the output end 26O. Therefore, the beam splitter 12 used to split the infrared ray I from the illuminating light L that is transmitted in the light-guide fiber 42 is not necessary.

In the present embodiment, light other then infrared ray I may also be used for generating electricity. That is, visible light or another light component may be transmitted by a fiber substituted for the infrared ray guide fiber 26. This is because the illuminating light L can be used only for illuminating the subject S, regardless of the light transmitted by the infrared ray guide fiber 26 and others that are independent from the light-guide fiber 42.

As explained above, in the present embodiment, the loss of infrared ray I in the splitting by the beam splitter 12, that is, the issue that a part of the infrared ray I does not enter the infrared ray absorber 14, can be reliably avoided. As a result, the efficiency of electricity generation by the peltier device 16 is increased. Furthermore, although the infrared ray guide fiber 26 is necessary, the beam splitter 12 is not required so the diameter of the insertion portion 40i of the scope 40 may be reduced.

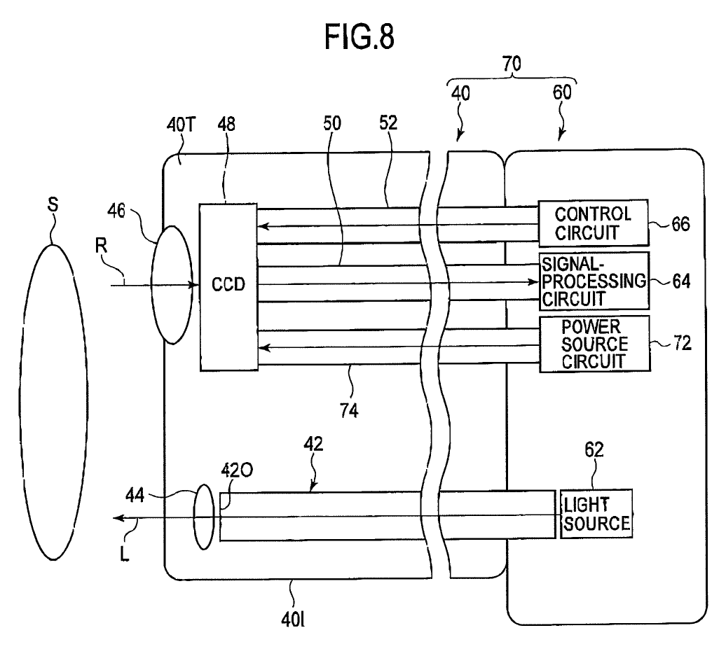

Next, a comparative example is explained with reference to FIG. 8. In an electronic endoscope 70 of the comparative example, the electrical power supply device 10 is not provided. Therefore, the electricity to drive the CCD 48 and other elements provided in the tip 40T is supplied by a power source circuit 72 in the processor 30 to the CCD 48 and others, via a power line 74.

Therefore, the power line 74 may act as an antenna to transmit or receive external noise, degrading the quality of a subject image, and possibly causing the CCD 48 to malfunction. Furthermore, because the power line 74 is provided, the diameter of the insertion portion 40I of the scope 40 will increase, and the insertion operation of the insertion portion 40I into the body of a subject may be more difficult.

On the other hand, in the above-explained embodiments in which the electrical power supply device 10 is provided, generation of noise by the power line 74 (see FIG. 8) is prevented, and the diameter of the insertion portion 40I of the scope 40 can be reduced. Furthermore, the visible light component of the illuminating light emitted by the light source 62 is not lost, so that the illuminating light can be efficiently utilized.

The members composing the electrical power supply device 10 are not limited to those in the embodiments. For example, instead of the peltier device 16, a thermoelectric semiconductor operating on the Seebeck effect could be used. Instead of the light source 62 that emits illuminating light L including components of visible light and infrared ray, a light source that emits only the visible light, another light source that emits only infrared ray, and a light combining prism that combines the visible light and infrared ray. In such a case, the infrared ray may be a laser light. Furthermore, the first and second beam splitters 12 and 13 may work in reverse fashion, so as to not reflect the first and second infrared ray I₁, I₂, but rather reflect the visible light and transmit the infrared ray. In such a case, the arrangements of the infrared ray absorber 14, peltier device 16, and solar cell 18 would be modified from those in the above-explained embodiments.

The invention is not limited to that described in the preferred embodiments; namely, various improvements and changes may be made to the present invention without departing from the spirit and scope thereof.

The present disclosure relates to subject matter contained in Japanese Patent Application No. 2007-191200 (filed on Jul. 23, 2007) which is expressly incorporated herein, by reference, in its entirety.

1. An electrical power supply device for an endoscope having a scope for insertion into the body of a subject, said electrical power supply device comprising:
a temperature controller that controls the temperature of the interior of said scope; and
a thermoelectric converter that generates electricity based on the difference in temperature between the body interior of the subject and the interior of said scope.

2. The electrical power supply device according to claim 1, wherein said temperature controller controls the temperature of the interior of said scope using the infrared component of illuminating light used to illuminate the body interior or the subject.

3. The electrical power supply device according to claim 2, further comprising a light splitter that splits said illuminating light, wherein said temperature controller comprises a photothermal converter that generates heat based on the infrared component that is split by said light splitter.

4. The electrical power supply device according to claim 2, further comprising a light-electric converter that generates electricity using part of said illuminating light.

5. The electrical power supply device according to claim 2, wherein said endoscope further comprises an optical fiber to transmit the infrared component to said scope.

6. The electrical power supply device according to claim 5, further comprising a light splitter that splits said illuminating light, wherein said light splitter is arranged close to the output end of said optical fiber.

7. The electrical power supply device according to claim 1, further comprising a secondary battery that is charged by the electricity generated by said thermoelectric converter.

8. The electrical power supply device according to claim 7, wherein said secondary battery is arranged inside said scope and is chargeable by electromagnetic coupling with the exterior of said scope.

9. The electrical power supply device according to claim 1, wherein said temperature controller comprises a cooler that cools the interior of said scope.

10. The electrical power supply device according to claim 1, wherein said temperature controller comprises a photothermal converter that generates heat from light and a light supply that supplies light to said photothermal converter.

11. An electrical power supply device for an endoscope having a scope for insertion into the body of a subject, said electrical power supply device comprising:
a first temperature controller that generates heat based on the infrared component or illuminating light used to illuminate the body interior of the subject;
a second temperature controller that cools the interior of said scope; and
a thermoelectric converter that generates electricity based on the difference in temperature between the body interior of the subject and the interior of said scope.

12. An endoscope comprising;
a scope for insertion into the body of a subject;
an imaging device;
a temperature controller that controls the temperature of the interior of said scope; and
a thermoelectric converter that generates electricity based on the difference in temperature between the body interior of the subject and the interior of said scope;
said imaging device driven by the electricity generated by said thermoelectric converter.

13. An endoscope comprising:
a scope for insertion into the body of the subject;
an imaging device;
a first temperature controller that generates heat based on the infrared component of illuminating light for illuminating the body interior of the subject;
a second temperature controller that cools the interior of said scope; and
a thermoelectric converter that generates electricity based on the difference in temperature between the body interior of the subject and the interior of said scope,
said imaging device driven by the electricity generated by said thermoelectric converter.
